(19) United States
(12) Patent Application Publication
(10) Pub. No.: US 2023/0116480 A1
(43) Pub. Date: Apr. 13, 2023

(54) SYSTEM AND METHOD FOR RECIPIENT-AWARE MESSAGE DELIVERY

(71) Applicant: Christiana Care Health System, Inc., Wilmington, DE (US)

(72) Inventors: Catherine Burch, Bear, DE (US); Jason Mastriana, Newark, DE (US)

(21) Appl. No.: 17/964,300

(22) Filed: Oct. 12, 2022

Related U.S. Application Data
(60) Provisional application No. 63/254,877, filed on Oct. 12, 2021.

(51) Int. Cl. H04L 51/226; G16H 20/70; G06V 40/10; G06V 40/16; G06V 40/70; G06V 10/764; G10L 25/63

(52) U.S. Cl. CPC H04L 51/226; G06V 10/764; G06V 40/15; G06V 40/70; G06V 40/174; G10L 25/63; G16H 20/70

(57) ABSTRACT

A recipient-aware message delivery system observes user activity via sensor hardware, such as a camera, microphone, or biometric or environmental condition sensor, and captures recipient activity data via the sensor. The activity data is analyzed to determine whether the recipient is in a stressed state, e.g., using facial and/or voice recognition, biometric and environmental data, etc. Messages intended for delivery to the recipient are processed. This may involve simply receiving a message intended for immediate delivery or determining whether the message content is likely to be stress-inducing. The system selectively delays delivery of messages to the recipient as a function of the recipient’s stress level by delivering messages without delay when the recipient is not in the stressed state, and by delaying before delivering if the recipient is in the stressed state. Accordingly, the system monitors a message recipient and intervenes when needed to prevent unnecessary stress.

### CROSS-REFERENCE TO RELATED APPLICATION

This application claims the benefit of priority of U.S. Provisional Pat. Application No. 63/254,877, filed Oct. 12, 2021, the entire disclosure of which is hereby incorporated herein by reference.

### FIELD OF THE INVENTION

The present invention relates generally to computerized systems, and more specifically, to a computerized system and method for recipient-aware message delivery that prevents unnecessary stress to the recipient and is conducive to the recipient’s health and well-being.

### DISCUSSION OF RELATED ART

In the modern world, many persons have extended and sometimes nearly continuous access, during waking hours, to computerized devices delivering messages and/or informational notifications, etc. to recipients. The barrage of emails, notifications, text messages, reminders, alerts, etc., have created habits that expose individuals to continuous information that directly adversely impacts a person’s health and well-being. For example, when an individual is already experiencing high levels of stress, receiving additional negative information via digital messaging can significantly adversely impact a person’s physical and behavioral health.

It is believed that the hyper-connectivity habits of individuals to technology and the unknown nature of digital messages (positive, negative, store offer, etc.) being received throughout the day effectively hijack us psychologically and affect us physiologically.

It is further believed that each individual experiences various triggers and levels of stress daily that can impact their overall health and well-being. Further, it is believed that managing stress levels can help individuals sleep better, avoid getting sick as often, and have less muscle tension. In addition, it is believed that various stress levels can increase the risk of obesity, heart disease, Alzheimer’s disease, diabetes, depression, gastrointestinal problems, and asthma.

Chronic stress from even minor nuisances over a period of time can cause lasting damage to both the mind and body, making a person feel fatigued, distracted, or irritable, and can even result in depression, increased risk of cardiovascular disease, headaches, heartburn, high blood pressure, and high blood sugar levels.

What is needed is a recipient-aware message delivery system that prevents unnecessary stress to the recipient and is conducive to the recipient’s health and well-being.

### BRIEF DESCRIPTION OF THE FIGURES

For a better understanding of the present invention, reference may be made to the accompanying drawings in which:

FIG. 1 is a system diagram showing an exemplary network computing environment in which the present invention may be employed;

FIG. 2 is a schematic block diagram illustrating an exemplary and non-limiting embodiment of a computerized Stress-Aware Analysis System in accordance with the present invention;

FIG. 3 is a schematic block diagram illustrating an exemplary and non-limiting embodiment of a computerized Stress-Aware Message Management System in accordance with the present invention; and

FIG. 4 is a flow diagram illustrating operation of the system in accordance with an exemplary and non-limited embodiment of the present invention.

### SUMMARY

The present invention relates generally to systems and methods for communication monitoring and/or intervention to prevent unnecessary stress, as may occur with hyper-connectivity in people checking devices. The present invention uses facial and/or voice recognition, biometric and/or environmental data, and machine learning to identify a person’s stress levels in real-time and determine whether to deliver immediately of an email, text message, reminder, alert, etc., or to delay delivery until a time that is more conducive to the person’s health and well-being.

More particularly, a recipient-aware message delivery system observes user activity via sensor hardware, such as a camera, microphone, or biometric or environmental condition sensor, and captures recipient activity data via the sensor. The activity data is analyzed to determine whether the recipient is in a stressed state, e.g., using facial and/or voice recognition, biometric and environmental data, etc. Messages intended for delivery to the recipient are processed. This may involve simply receiving a message intended for immediate delivery or analyzing the message to determine whether the message content is likely to be stress-inducing (or not stress-inducing). The system selectively delays delivery of messages to the recipient as a function of the recipient’s stress level by delivering messages without delay when the recipient is not in the stressed state, and by delaying before delivering if the recipient is in the stressed state. Accordingly, the system monitors a message recipient and intervenes when needed to prevent unnecessary stress.

### DETAILED DESCRIPTION

The present invention provides a computerized system including hardware for recipient and/or recipient communication monitoring and/or intervention to prevent unnecessary stress, as may occur with hyper-connectivity in people checking electronic devices. The ambient productivity system and method may use one or more of facial and voice recognition, biometric and environmental data, and machine learning to identify a recipient’s stress levels in real-time and resultingly determine when to deliver an email, text message, reminder, notification, alert, etc., namely, at a time that is conducive to the person’s health and well-being, e.g., between or after periods of observed/deduced heightened stress.

A system in accordance with the present invention may use hardware and software used for facial and voice recognition, biometric and environmental data, and machine learning to determine in real-time an individual’s current stress levels and/or message content. This information may be used to determine when not to deliver a message that would otherwise be delivered, and instead, to delay delivery of a message to another time, preferably one in which the intended recipient is determined or expected to have a lower stress level.

For example, the system may use native sensors and inputs from IoT devices, including facial and voice analysis, biometric and environmental data, etc. The system pulls the information into the Emotive Processing Interface Engine (EPIE) existing on local devices and/or within the cloud to analyze and determine users’ current stress levels in real-time. These results may be stored securely in the EPIE secure storage database.

A system in accordance with the present invention may use hardware and software to determine the nature/content of an incoming email, notification, text message, reminder, alert, etc., and determine if the content will have a negative impact on the person’s health and well-being.

For example, during a period of high recipient stress, no messages may be delivered, or only urgent messages may be delivered, or only messages determined not to be stressful may be delivered. Other messages may be delayed, e.g., for a defined period of time, or until a time in which a lower stress level of the recipient is detected, or until a time at which the recipient is expected to have a lower stress level, e.g., as determined from routine, machine learning/AI, habits, patterns, etc.

By way of further example, if the system identifies a non-emergent stressful email, notification, text message, reminder, etc., during a period of high stress of the recipient, then the system may delay delivering that message, etc. based on a user or system setting/preference.

Device operating systems (mobile, desktop, wearables, etc.) may access the EPIE secure storage database in real-time according to a predetermined ambient productivity algorithm to evaluate and determine if incoming messages and alerts can be immediately delivered or delayed based on the users’ preference settings including, words, phrases, senders, subject, images, length, videos, audio, attachments, date/time, formatting, etc. that impact a user’s stress levels and health and well-being.

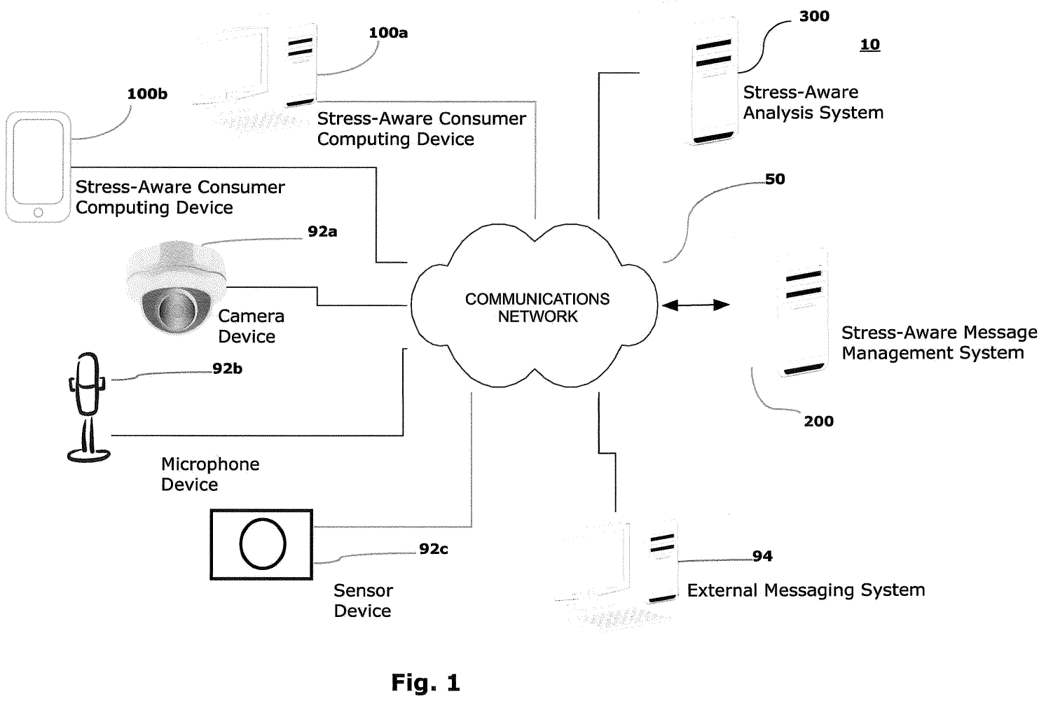

According to illustrative embodiment(s) of the present invention, various views are illustrated in FIGS. 1-4 and like reference numerals are used consistently throughout to refer to like and corresponding parts of the invention for all of the various views and figures of the drawings.

The following detailed description of the invention contains many specifics for the purpose of illustration. Anyone of ordinary skill in the art will appreciate that many variations and alterations to the following details are within the scope of the invention. Accordingly, the following implementations of the invention are set forth without any loss of generality to, and without imposing limitations upon, the claimed invention.

### System Environment

An exemplary embodiment of the present invention is discussed below for illustrative purposes. FIG. 1 is a system diagram showing an exemplary network computing environment 10 in which the present invention may be employed. As shown in FIG. 1, the exemplary network environment 10 includes certain conventional computing hardware and software for communicating via a communications network 50, such as the Internet, etc., using a Stress-Aware Consumer Computing Device (SACCD) 100a, 100b, each of which may be, for example, one or more personal computers/PCs, laptop computers, tablet computers, smartphones, or other computing system hardware, including computerized/networked communication hardware/software/functionality, such as computer-based servers, kiosks and the like, or other so-called “connected” communication devices having communication capabilities for communicating data via the network and for delivery messages, in any form, to the user, such as smart watches, activity trackers, headphones, ear buds, televisions, or any other computerized and/or internet-of-things type device. By way of example, “messages” is used in a broad non-limiting fashion to include messages deliverable a user via such a communication device, such as an e-mail message, a text/SMS or similar “i-message”, as well as notifications such as a telephone ring, beep/tone or other audible or haptic alert, calendar reminders, alert messages and other displayable textual or graphic notifications designed to notify and/or communicate information to a user of the communication device.

In accordance with a certain aspect of the present invention, one or more of the Stress-Aware Consumer Computing Devices (SACCDs) 100a, 100b is a smartphone, tablet computer, smart watch or other computing device configured to store and execute an “app” or other purpose-specific application software in accordance with the present invention, although this is not required in all embodiments.

In accordance with another aspect of the present invention, the exemplary network environment 10 includes certain conventional sensor hardware 92a, 92b, 92c capable of observing the activities of a user and capturing associated data and electronically communicating it to another device, e.g., via the communications network 50. More particularly, sensor hardware may be employed that is capable of capturing user activity data that can be interpreted to determine whether or not the user is in a stressed state, e.g., using conventional analytical techniques. For example, one or more heart rate sensors may be used to monitor a heart rate of a person, and a stress state may be determined by heart rate variability, according to the monitored heart rate. Additional, examples of such conventional hardware includes a camera device 92a (which may be used for example to capture facial image data indicative of a stressed state), a microphone device 92b (which may be used for example to capture vocalizations indicative of a stressed state), and other sensor devices 92c, such as a biometric sensor for capturing fingerprint, face, iris, vein data indicative of a stressed state, or other sensors for capturing activity and/or environmental data indicative of a stressed state, etc., such as those capturing data related to aspects of keystroke dynamics, gait, signature, etc. In certain embodiments, these sensors may be an integral part of the Stress-Aware Consumer Computing device 100a, 100b. In other embodiments, these sensors may be separate from the SACCDs 100a, 100b.

In accordance with another aspect of the present invention, the exemplary network environment 10 further includes an External Messaging System (EMS) 94. By way of example, the EMS 94 may be a purely conventional electronic mail server or text message server configured to send e-mail or text messages to consumer computing and/or communication devices. In certain embodiments, the EMS 94 may be modified to include software in accordance with the present invention to provide structure and functionality described herein a residing elsewhere in the exemplary network computing environment 10.

Hardware and software for enabling communication of data by such systems via such communications networks are well known in the art and beyond the scope of the present invention, and thus are not discussed in detail herein.

### Stress-Aware Analysis System

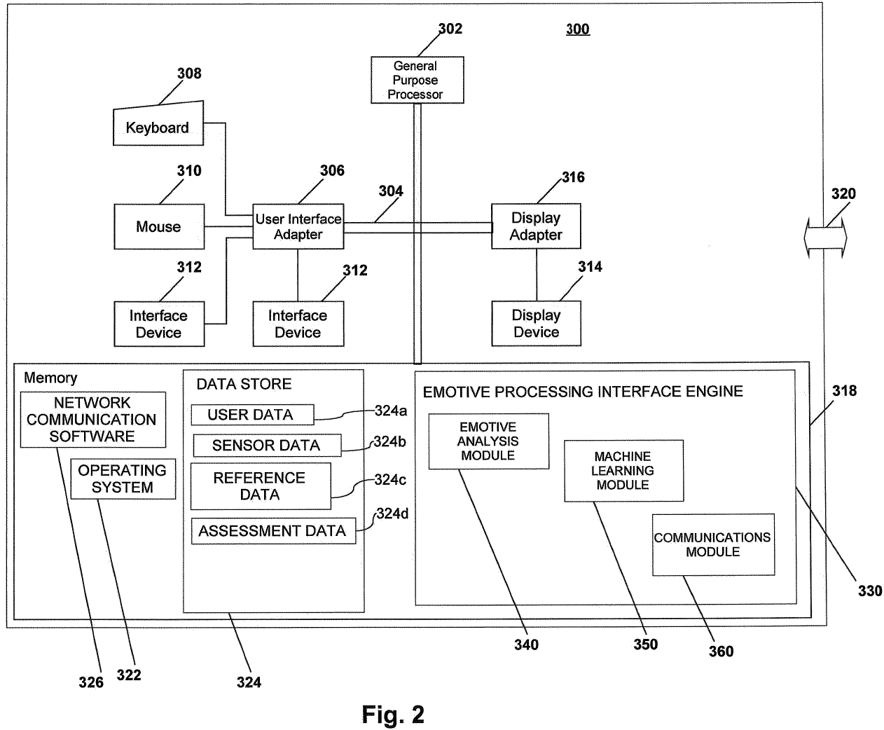

FIG. 2 is a schematic block diagram showing an exemplary Stress-Aware Analysis System (SAAS) 300 in accordance with an exemplary embodiment of the present invention. The SAAS 300 is a special-purpose computer system that includes conventional computing hardware storing and executing both conventional software enabling operation of a general-purpose computing system, such as operating system software, network communications software, and specially-configured computer software for configuring the general purpose hardware as a special-purpose computer system for carrying out at least one method in accordance with the present invention. By way of example, the communications software may include conventional web server software, and the operating system software may include iOS, Android, Windows, Linux software.

Referring again to FIG. 2, there is illustrated a block diagram of an exemplary SAAS 300 according to some embodiments is shown. In some embodiments, the SAAS 300 may, for example, execute, process, facilitate, and/or otherwise be associated with the embodiments described above.

Accordingly, the exemplary SAAS 300 of FIG. 2 includes a general-purpose processor, such as a microprocessor (CPU), 302 and a bus 304 employed to connect and enable communication between the processor 302 and the components of the presentation system in accordance with known techniques. According to some embodiments, the processor 302 may be or include any type, quantity, and/or configuration of processor that is or becomes known. In some embodiments, the processor 302 may comprise multiple inter-connected processors, microprocessors, and/or micro-engines. According to some embodiments, the processor 302 (and/or the system 300 and/or other components thereof) may be supplied power via a power supply (not shown), such as a battery, an Alternating Current (AC) source, a Direct Current (DC) source, an AC/DC adapter, solar cells, and/or an inertial generator. In the case that the system 300 comprises a server, such as a blade server, necessary power may be supplied via a standard AC outlet, power strip, surge protector, and/or Uninterruptible Power Supply (UPS) system.

The exemplary SAAS 300 includes a user interface adapter 306, which connects the processor 302 via the bus 304 to one or more interface devices, such as a keyboard 308, mouse 310, and/or other interface devices 312, which can be any user interface device, such as a touch-sensitive screen, digitized entry pad, etc. The bus 304 also connects a display device 314, such as an LCD screen or monitor, to the processor 302 via a display adapter 316.

The bus 304 also connects the processor 302 to memory 318, which can include a hard drive, a solid-state drive, an optical drive, a diskette drive, a tape drive, etc. The memory 318 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 318 may, according to some embodiments, store one or more software components. Any or all of the exemplary instructions and data types described herein and other practicable types of data may be stored in any number, type, and/or configuration of memory systems that is or becomes known. The memory 318 may, for example, comprise one or more data tables or files, databases, table spaces, registers, and/or other storage structures. In some embodiments, multiple databases and/or storage structures (and/or multiple memory systems) may be utilized to store information associated with the system 300. According to some embodiments, the memory 318 may be incorporated into and/or otherwise coupled to the system 300 (e.g., as shown) or may simply be accessible to the system 300 (e.g., externally located and/or situated).

The SAAS 300 may communicate with other computers or networks of computers, for example via a communications channel, network card, modem or transceiver (collectively, “transceiver”) 220. In some embodiments, the transceiver 220 may comprise any type or configuration of communication system that is or becomes known or practicable. The transceiver 220 may, for example, comprise a Network Interface Card (NIC), a telephonic system, a cellular network system, a router, a hub, a modem, and/or a communications port or cable. According to some embodiments, the transceiver 220 may also or alternatively be coupled to the processor 302. In some embodiments, the transceiver 220 may comprise an IR, RF, Bluetooth™, Near-Field Communication (NFC), and/or Wi-Fi® network system coupled to facilitate communications between the processor 302 and another system (not shown). The SAAS 300 may be associated with such other computers in a local area network (LAN) or a wide area network (WAN), and may operate as a server in a client/server arrangement with another computer, etc. Such configurations, as well as the appropriate communications hardware and software, are known in the art.

The SAAS 300 is specially configured in accordance with the present invention. Accordingly, as shown in FIG. 2, the SAAS includes computer-readable, processor-executable instructions stored in the memory 318 for carrying out the methods described herein. Further, the memory 318 stores certain data, e.g., in one or more databases or other data stores 324 shown logically in FIG. 2 for illustrative purposes, without regard to any particular embodiment in one or more hardware or software components.

Further, as will be noted from FIG. 2, the SAAS 300 includes, in accordance with the present invention, an Emotive Processing Interface Engine (EPIE) 330, shown schematically as stored in the memory 318, which includes a number of additional modules providing functionality in accordance with the present invention, as discussed in greater detail below. These modules may be implemented primarily by specially-configured software including microprocessor-executable instructions stored in the memory 318 of the SAAS 300. Optionally, other software may be stored in the memory 318 and and/or other data may be stored in the data store 324 or memory 318. Further, the EIPE 230 includes one or more modules shown logically in FIG. 2 for illustrative purposes, without regard to any particular embodiment in one or more hardware or software components.

It should be noted that some of the wording and form of description herein is done to meet applicable statutory requirements. Although the terms “step”, “block”, “module”, “engine”, etc. might be used herein to connote different logical components of methods or systems employed and/or for ease of illustration, the terms should not be interpreted as implying any particular order among or between various steps herein disclosed unless and except when the order of individual steps is explicitly described, or be interpreted as implying any distinct structure separate and apart from other structures of the system.

As shown in FIG. 2, the SAAS 300 includes a data store 224 and an Emotive Processing Interface Engine (EPIE) 230 in accordance with the present invention. The EPIE is operable to receive user data and/or sensor-obtained data that may be used to determine whether or not the user is in a stressed state, as discussed in greater detail below.

In part, the SAAS 300 stores User Data 224a in the data store 224, e.g., in a database cluster. The User Data 224a identifies the user and includes any relevant user-identified and user-associated data, such as contact and communication information, including information for transmitting data to and/or otherwise interfacing with the user’s Stress-Aware Consumer Computing Device, if needed. By way of example, some or all of this information may be provided by or gathered from the user by direct input or by data communication via the network 50 with the user’s Stress-Aware Consumer Computing Device 100a, 100b.

Further, the SAAS 300 stores Sensor Data 224b in the data store 224. The Sensor Data 224b is data gathered by sensor devices that are used to identify activities or other aspects associated with the user and relevant to determining whether the user is presently in a stressed state. For example, the Sensor Data 224b may include data captured by a camera device 92a (which may be used for example to capture facial image data indicative of a stressed state), a microphone device 92b (which may be used for example to capture vocalizations indicative of a stressed state), and other sensor devices 92c, such as a biometric sensor for capturing fingerprint, face, iris, vein data indicative of a stressed state, or other sensors for capturing activity and/or environmental data indicative of a stressed state, etc., such as those capturing data related to aspects of keystroke dynamics, gait, signature, etc. In certain embodiments, these sensors may be an integral part of the Stress-Aware Consumer Computing Device 100a, 100b. In other embodiments, these sensors may be separate from the SACCDs 100a, 100b.

The EPIE 230 includes an Emotive Analysis Module (EAM) 240 that is operable to receive and/or retrieve User Data 224a and/or Sensor Data 224b from the data store 224 and to analyze such data to make a determination as to whether or not the user is in a stressed state (or an unstressed state) based on one or more of the User Data 224a and the Sensor Data 224b. In certain embodiments, this may involve comparison to Reference D224c stored in the data store 224 in the memory 218. Any suitable methodology may be used to analyze the relevant data and make a determination as to whether or not the user is in a stressed state as a result of the data. For example, for a given user, current facial image or vocalization data may be compared to previously-stored facial image and vocalizations for that user, or to reference image and/or vocalization data, to determine similarities and/or differences supporting a conclusion to that the user is in a stressed state. By way of further example, a blood pressure or temperature sensor may capture current blood pressure or temperature data as Sensor D324b and compare that to the user’s own at-rest blood pressure and temperature data stored as User Data 224b and/or to reference blood pressure and temperature data stored as Reference Data 224c. By way of example, known facial recognition, audio signal analysis, and biometric techniques may be used to make this determination.

Additionally, the data store 224 stores User Assessment Data 224d. The User Assessment Data 224d is the output from the EAM 340, and reflects whether or not the associated user is in a stressed state, so that the user state information may be used for the purposes described herein.

In certain embodiments, the EPIE 230 further includes a Machine Learning Module (MLM) 250. The MLM 250 is operable to analyze sensor data, user data, reference data, and/or assessment data, and to apply machine learning techniques to improve the accuracy of assessments (i.e., determinations of whether or not users are in a stressed state). By way of example, a heart rate may be monitored and heart rate data may be stored as relevant heart rate reference data and may be referenced to identify heart rate variability that is deemed to indicate a stressed state, and the MLM may be used to determine, for a particular user a heart rate or heart rate variability that indicates a normal/unstressed state, rather than a stressed state.

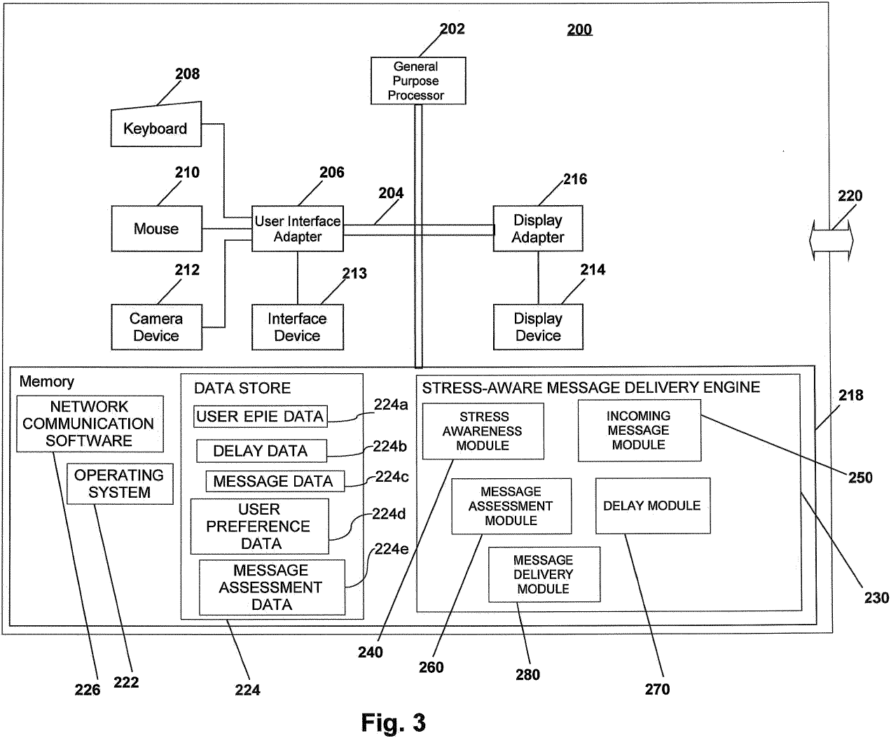

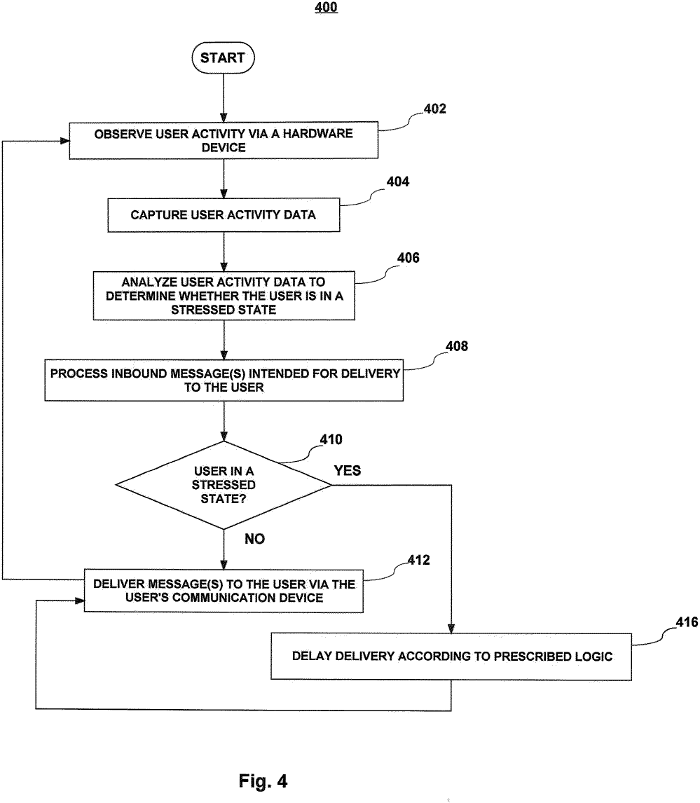

The EPIE 230 further includes a Communications Module (CM) 260 that is operable to communicate (e.g., transmit data to an internal component within a single computerized device or across a communications network to another computing device) data indicating whether or not a particular user is in a stressed state, e.g., based on the Assessment Data 224d output from the EAM 240. In certain embodiments, this may involve transmission of data from the SAAS 300 to a Stress-Aware Consumer Computing Device 100a, 100b (e.g., where the some or all of the structure and/or functionality of FIGS. 3 and 4 are present at a Stress-Aware Consumer Computing Device 100a, 100b) or a Stress-Aware Message Management System 200 for the purposes described below.

### Stress-Aware Message Management System

FIG. 3 is a schematic block diagram showing an exemplary Stress-Aware Message Management System (SAMMS) 200 in accordance with an exemplary embodiment of the present invention. The SAMMS 200 is a special-purpose computer system that includes conventional computing hardware storing and executing both conventional software enabling operation of a general-purpose computing system, such as operating system software, network communications software, and specially-configured computer software for configuring the general purpose hardware as a special-purpose computer system for carrying out at least one method in accordance with the present invention. By way of example, the communications software may include conventional web server software, and the operating system software may include iOS, Android, Windows, Linux software.

Referring again to FIG. 3, there is illustrated a block diagram of an exemplary SAMMS 200 according to some embodiments is shown. In some embodiments, the SAMMS 200 may, for example, execute, process, facilitate, and/or otherwise be associated with the embodiments described above.

Accordingly, the exemplary SAMMS 200 of FIG. 3 includes a general-purpose processor, such as a microprocessor (CPU), 202 and a bus 204 employed to connect and enable communication between the processor 202 and the components of the presentation system in accordance with known techniques. According to some embodiments, the processor 202 may be or include any type, quantity, and/or configuration of processor that is or becomes known. In some embodiments, the processor 202 may comprise multiple inter-connected processors, microprocessors, and/or micro-engines. According to some embodiments, the processor 202 (and/or the system 200 and/or other components thereof) may be supplied power via a power supply (not shown), such as a battery, an Alternating Current (AC) source, a Direct Current (DC) source, an AC/DC adapter, solar cells, and/or an inertial generator. In the case that the system 200 comprises a server, such as a blade server, necessary power may be supplied via a standard AC outlet, power strip, surge protector, and/or Uninterruptible Power Supply (UPS) system.

The exemplary SAMMS 200 includes a user interface adapter 206, which connects the processor 202 via the bus 204 to one or more interface devices, such as a keyboard 208, mouse 210, camera device 212 and/or other interface devices 214, which can be any user interface device, such as a microphone, biometric sensor, touch sensitive screen, digitized entry pad, etc. The bus 204 also connects a display device 214, such as an LCD screen or monitor, to the processor 202 via a display adapter 216.

The bus 204 also connects the processor 202 to memory 218, which can include a hard drive, a solid-state drive, an optical drive, a diskette drive, a tape drive, etc. The memory 218 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 218 may, according to some embodiments, store one or more software components. Any or all of the exemplary instructions and data types described herein and other practicable types of data may be stored in any number, type, and/or configuration of memory systems that is or becomes known. The memory 218 may, for example, comprise one or more data tables or files, databases, table spaces, registers, and/or other storage structures. In some embodiments, multiple databases and/or storage structures (and/or multiple memory systems) may be utilized to store information associated with the system 200. According to some embodiments, the memory 218 may be incorporated into and/or otherwise coupled to the system 200 (e.g., as shown) or may simply be accessible to the system 200 (e.g., externally located and/or situated).

The SAMMS 200 may communicate with other computers or networks of computers, for example via a communications channel, network card, modem or transceiver (collectively, “transceiver”) 220. In some embodiments, the transceiver 220 may comprise any type or configuration of communication system that is or becomes known or practicable. The transceiver 220 may, for example, comprise a Network Interface Card (NIC), a telephonic system, a cellular network system, a router, a hub, a modem, and/or a communications port or cable. According to some embodiments, the transceiver 220 may also or alternatively be coupled to the processor 202. In some embodiments, the transceiver 220 may comprise an IR, RF, Bluetooth™, Near-Field Communication (NFC), and/or Wi-Fi® network system coupled to facilitate communications between the processor 202 and another system (not shown). The SAMMS 200 may be associated with such other computers in a local area network (LAN) or a wide area network (WAN), and may operate as a server in a client/server arrangement with another computer, etc. Such configurations, as well as the appropriate communications hardware and software, are known in the art.

The SAMMS 200 is specially configured in accordance with the present invention. Accordingly, as shown in FIG. 3, the SAMMS includes computer-readable, processor-executable instructions stored in the memory 218 for carrying out the methods described herein. Further, the memory 218 stores certain data, e.g., in one or more databases or other data stores 224 shown logically in FIG. 3 for illustrative purposes, without regard to any particular embodiment in one or more hardware or software components.

Further, as will be noted from FIG. 3, the SAMMS 200 includes, in accordance with the present invention, a Stress-Aware Message Delivery Engine (SAMDE) 230, shown schematically as stored in the memory 218, which includes a number of additional modules providing functionality in accordance with the present invention, as discussed in greater detail below. These modules may be implemented primarily by specially-configured software including microprocessor-executable instructions stored in the memory 218 of the SAMMS 200. Optionally, other software may be stored in the memory 218 and and/or other data may be stored in the data store 224 or memory 218. Further, the SAMDE 230 includes one or more modules shown logically in FIG. 3 for illustrative purposes, without regard to any particular embodiment in one or more hardware or software components.

As shown in FIG. 3, the SAMMS 200 includes a data store 224 and a Stress-Aware Message Delivery Engine (SAMDE) 230 in accordance with the present invention. The SAMDE is operable to receive user stress state data from the EPIE 330 of the SAAS 300, as well as receive, process and/or otherwise cause selective delay of delivery of messages depending upon the user stress state and/or the quality/content of the messages, as discussed in greater detail below.

In part, the SAMMS 200 stores User EPIE Data 224a in the data store 224, e.g., in a database cluster. The User EPIE Data 224a may be received from the EPIE 330 of the SAAS 300 (e.g., as Assessment Data 324d communicated to the SAMMS 200 by the Communication Module 360 of the EPIE 330. The User EPIE Data 224a identifies a particular user’s stress state to indicate whether the user has been determined presently/recently to be in a stressed state (or an unstressed state), e.g., as determined by the SAAS using activity/sensor data associated with the user, as described above.

Further, the SAMMS 200 stores Delay Data 224b in the data store 224. The Delay Data may be settings, preferences and/or logic for delay of messages prior to delivery in the event that the user is in a stressed state. The Delay Data may be established at the system level for all users, for groups of users, or on a per-user level, and may be a system-established setting or a user-specified setting. For example, the Delay Data may provide for a time delay - e.g., to delay for a specified period of time, or until a certain time frame, e.g., next business day’s business working hours. Alternatively, the Delay Data may provide for a stress state delay - e.g., to delay until the user is no longer detected as being in a stressed state.

The SAMDE 230 includes a Stress Awareness Module (SAM) 240 that is operable to receive and/or retrieve User EPIE Data 224a providing a determination as to whether or not the user is in a stressed state (or an unstressed state). This may happen periodically, sporadically, and/or continuously or substantially continuously, according to any suitable preference.

The SAMDE 230 includes an Incoming Message Module (IMM) 250 that is operable to receive and/or retrieve incoming messages intended for display or other delivery (e.g., as an audible sound) to the user. The incoming messages may be any form of message (e.g., e-mail or text, etc.) or notification (e.g., alarm, notification tone, operating system banner message, graphic, etc.) intended for delivery to a user that may serve to stimulate the user and cause stress or increased stress in the user. The IMM 250 may store incoming messages as Message Data 224c in the Data Store 224. Accordingly, the IMM 250 effectively provides an intermediate process for monitoring, processing and/or intercepting a message prior to conventional delivery to a user, for the purposes described herein.

In the exemplary embodiment of FIG. 3, the SAMDE 230 further includes an optional Message Assessment Module (MAM) 260 that is operable to process/analyze incoming messages prior to delivery, to determine whether (or not) they are likely to cause stress or increased stress to a user. This may be performed in any suitable manner. By way of example, it may be determined by the presence (or absence) of an “urgent” or “high priority” flag associated with the message. Alternatively, it may be determined by scanning text of the message and matching words or capitalization patterns with a predetermined word list deemed to be indicative of a stressful message (e.g., inclusion of the words/phrases “immediately”, “hate,” “shut up”, profane words, certain phrases, etc., or according to the sender of the message, the subject of the message, the date/time of sending of the message, the formatting of the message, the content of included audio, video, or textual attached files, the names of attached files, etc.). The MAM 260 may store its assessment of the message (e.g., indicating it as stress-inducing or not stress-inducing) as Message Assessment Data 224e in the Data Store 224. Accordingly, the MAM 260 effectively provides an intermediate process for processing a message prior to conventional delivery to a user, for the purposes described herein.

The SAMDE 230 further includes a Delay Module (DM) 270 that is operable to selectively delay delivery of messages to user according to the determination of the user’s stress state (e.g., stressed or unstressed). More particularly, the Delay Module 270 interacts with the Stress Awareness Module 240 to determine whether the user is in a stressed state, and with the Incoming Message Module to determine whether there is a message intended for delivery to the user. In certain embodiments, the Delay Module 270 causes display of a graphical user interface to the user via a display device of the User’s Consumer Computing Device to gather user input that it stores as user Preference Data 224d to indicate whether and/or how a user would like to receive messages. For example, this may include User Preferences as to whether or not to delay messages and logic for doing so (in addition to any settings in the Delay Data 224b). Additionally, the Delay Module 270 may gather user input as to whether and/or how the user would like to delay messages based on the system’s assessment of message content. In such an embodiment, the Delay Module 270 also interacts with the Message Assessment Module and its assessment of messages/ Message Assessment Data 224e in making its determination of whether and/or how to delay delivery of each message intended for delivery to the user. The Delay Module 270 imposes the appropriate delay, and then interacts with the Message Delivery Module 280.

The SAMDE 230 further includes a Message Delivery Module (MDM) 280 that is operable to deliver or cause delivery of the message. Delivery of the message may be affected in the conventional fashion after initiation of delivery by the MDM 280. The Delay Module 270 imposes the appropriate delay (as described above), and then interacts with the Message Delivery Module 280, which in turn causes delivery of the message after initiation of delivery by the DM 270. This causes the message (delayed or not according to the determinations of the Delay Module 270) to then be displayed or otherwise delivered to the user via the user’s Stress-Aware Consumer Computing Device 100a, 100b. By way of example, the MDM 280 may cause display of the message to the user by forwarding the messages from the SAAMS 200 to the User’s Stress-Aware Consumer Computing Device 100a, 100b, or may involve interacting the with Operation System or application software to cause display/delivery of a messages already received by the Stress-Aware Consumer Computing Device 100a, 100b.

### System Operation

Exemplary operation of the system of FIGS. 1-3 is discussed below with reference to the flow diagram 400 of FIG. 4.

Referring now to FIG. 4, it will be appreciated that an exemplary method of operation of a system in accordance with the present invention involves the system observing user activity via a hardware device, as shown at 402. This may involve, for example, use of a camera device 92a to observe a user’s face, a microphone device 92b, to capture a user’s vocalizations, or any suitable sensor 92 to capture user biometric information, user activity information, or user environmental information (e.g., temperature, etc.). Notably, the hardware device used for this purposes may be a discrete device as shown with reference to camera device 92a, microphone device 92b, and sensor device 92c, or may be any hardware device integrated into another machine or device, such as a camera device, microphone device or other sensor device integrated into a user’s smartphone, tablet computer, laptop computer, desktop computer or other Stress-Aware Consumer Computing device 100a, 100b.

The exemplary method next involves capturing user activity data using such hardware device, as shown at 404. For example, this may involve capturing a facial image, a voice/vocalization sample, a temperature reading, etc. Any appropriate data (referred to as User Activity Data in FIG. 4) may be captured using any suitable sensor, and may be stored and communicated to (e.g., via the communications network 50) and stored by the SAAS 300 as sensor Data 324b, e.g., along with associated user-identifying User data 324a (FIG. 2).

The exemplary method next involves analyzing the user activity data to determine whether the user in in a stressed state, as shown at 406. As discussed above, this may be performed by the EPIE 330 (more particularly by the EAM 340, with or without assistance of the MEM 350) of the SAAS 300, e.g., by referring to the Sensor Data 324b and/or Reference Data 324c., and storing the determination of the user’s stress state (stressed and/or unstressed) as Assessment Data 324d. The current stress determination for the user is then communicated to the SAMMS 200 (for monitoring to determine the current stress state by the SAM 240) by the Communications Module 360 of the EPIE 330 for use in determining whether to delay delivery of messages according to the user’s stress state, as described above.

Notably, steps 402-406 may be performed repeatedly according to any desired schedule or regime to provide a suitably up-to-date insight as to whether the user is currently in a stressed or unstressed state, such that messages may be delayed or delivered according to the teachings herein.

The exemplary method next involves processing one or more inbound messages intended for delivery to the user, as shown at 408. This may involve the Incoming Message Model 250 of the SAMMS 200 receiving/noting the message’s availability for delivery and/or may involve in certain embodiments the Message Assessment Module 260 analyzing the message content or characteristics (collectively “content”) to classify the message as stress-inducing or not-stress-inducing for content-based delay of messages, as described above.

The exemplary method next involves determining whether the user is in a stressed state, as shown at 410. This may involve the Delay Module 270 interacting with the Stress Awareness Module 240 to ascertain the user’s current stress state.

If the Delay Module 270 determines that the user is not currently in a stressed state (e.g., by interfacing with the SAM 240, which may reference the EPIE Data 224a received from the Stress-Aware Analysis System 300), then the Delay Module 270 may interact with the Message Delivery Module 280 to cause delivery of the message to the user without delay, as shown at 412, and method flow may return to monitoring user activity as shown at 402. Accordingly, if the user is not determined to presently be in a stressed state, then incoming messages may be delivered to the user in the usual fashion. In this case, the processing of the messages for possible-delayed delivery may be “transparent” or otherwise generally unnoticeable to the user.

If, however, the Delay Module 270 determines that the user is currently in a stressed state (e.g., by interfacing with the SAM 240, which may reference the EPIE Data 224a received from the Stress-Aware Analysis System 300), then the Delay Module 270 may interact with the Message Delivery Module 280 to delay/postpone delivery of the message to the user, as shown at 416. For example, this may involve delaying for a prescribed period of time for all messages, or delaying until a particular timeframe for all messages, or delay until the user is no longer is a stressed state for all messages, or delaying according to any suitable regime only for selected messages determined to have content/characteristics that are determined to be stress-inducing and to therefore be likely to cause or increase the user’s stress level, as described above. After delaying delivery according to any suitable logic at 416, method flow then returns to 412 to cause the incoming message(s) to be delivered to the user in the usual fashion, as described above, and method flow then returns to 402 for further observation of user activity via a hardware device in support of making determination of the user’s current stress state, as described above.

It should be appreciated that the exemplary embodiment described above is for illustrative purposes only, and non limiting. For example, certain functionality was described above for illustrative clarity in relation to functions performed at the Stress-Aware Computing Devices 100a, 100b, SAAS 300 and SAMMS 200 separately. However, it should be appreciated that in other embodiments, some or all of the structure and functionality described in relation to each of the Stress-Aware Computing Devices 100a, 100b, SAAS 300 and SAMMS 200 may be instead be incorporated into another one of the Stress-Aware Computing Devices 100a, 100b, SAAS 300 and SAMMS 200. For example, the functionality of the SAAS 300 may be incorporated in whole or in part into either the Stress-Aware Computing Devices 100a, 100b and/or the SAMMS 200. By way of further example, the functionality of the SAMMS 200 may be incorporated in whole or in part into the SAAS 300 and/or the Stress-Aware Computing Devices 100a, 100b. By way of further example, all of the functionality of the SAAS 300 and the SAMMS 200 may be incorporated into the Stress-Aware Computing Devices 100a, 100b, etc.

Accordingly, messages are not merely delivered as received/ready for delivery, or according to a predetermined schedule, or according to the time sent, but rather are selectively delivered and/or delayed according to the recipient’s current or potential stress levels in real-time, to avoid adverse impacts to the recipient’s health and well-being resulting from receipt of the messages. By way of further example, a non-emergent stressful email, notification, text message, reminder, etc., may be delayed during a period of high stress of the recipient. In that case, the system will delay delivering that message, e.g., based on a user preference setting. By way of further example, a message determined to be stressful but urgent (e.g., with an urgent/high priority flag) may not be delayed during a period of high stress of the recipient, or it may be delayed, based on a user preference setting

Accordingly, the present invention can benefit individuals within any age group, who are healthy, who suffer from underlying conditions, or who would like a way to manage daily stress levels. This present invention provides a proactive option for individuals who desire another layer for managing daily stress and can help coach and guide individuals for better technology habits that are beneficial to the individual’s health and well-being.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A recipient-aware message delivery system comprising:
a memory operatively comprising a non-transitory data processor-readable medium;
a data processor operatively connected to the memory;
sensor hardware operatively connected to the data processor and operable to capture recipient user activity data relating to a stress level of a message recipient;
user interface management instructions embodied in data processor-executable code stored in the memory, said user interface management instructions being executable by the data processor to provide a stress-aware message delivery engine configured to:
observe recipient user activity via the sensor hardware;
capture recipient user activity data via the sensor hardware;
analyze the recipient user activity data to determine whether the recipient user is in a stressed state;
process at least one message intended for delivery to the recipient user; and
selectively delaying delivery of said at least one message to the recipient as a function of the recipient’s stress level by:
delivering said at least one message without delay if the recipient user is determined not to be in the stressed state; and
delaying and delivering said at least one message after delay if the recipient user is determined to be in the stressed state.

2. The recipient-aware message delivery system of claim 1, wherein said sensor hardware comprises a camera device and wherein said stress-aware message delivery engine is configured to observe recipient user activity via said camera device and use at least one of facial recognition and machine learning to determine whether a facial image captured by the camera device indicates that the recipient is in the stressed state.

3. The recipient-aware message delivery system of claim 1, wherein said sensor hardware comprises a microphone device and wherein said stress-aware message delivery engine is configured to observe recipient user activity via said microphone device and use at least one of voice analysis and machine learning to determine whether a vocalization captured by the microphone device indicates that the recipient is in the stressed state.

4. The recipient-aware message delivery system of claim 1, said sensor hardware comprises a biometric device and wherein said stress-aware message delivery engine is configured to observe recipient user activity via said biometric device and determine whether biometric data captured by the biometric device indicates that the recipient is in the stressed state.

5. The recipient-aware message delivery system of claim 4, wherein said biometric device is a heart rate sensor and wherein said stress-aware message delivery engine is configured to determine whether recipient is in the stressed state as a function of heart rate variability.

6. The recipient-aware message delivery system of claim 1, said sensor hardware comprises an environmental sensor device and wherein said stress-aware message delivery engine is configured to observe recipient user activity via said environmental sensor device and determine whether environmental data captured by the biometric device indicates that the recipient is in the stressed state.

7. The recipient-aware message delivery system of claim 1, wherein said stress-aware message delivery engine is configured to analyze the recipient user activity data to determine whether the recipient user is in a stressed state using machine learning by reference to previously-observed reference data.

8. The recipient-aware message delivery system of claim 1, wherein said stress-aware message delivery engine is configured to process at least one message intended for delivery to the recipient user by identifying said at least one message as a message otherwise ready for delivery the recipient user.

9. The recipient-aware message delivery system of claim 1, wherein said stress-aware message delivery engine is configured to process at least one message intended for delivery to the recipient user by analyzing at least one of content and characteristics of said at least one message to determine whether said at least one message is stress-inducing.

10. The recipient-aware message delivery system of claim 9, wherein said stress-aware message delivery engine is configured to delay only if said analyzing of said at least one of content and characteristics of said at least one message is determined to be stress-inducing.

11. The recipient-aware message delivery system of claim 9, wherein said stress-aware message delivery engine is configured to deliver said at least one message without delay even if said analyzing of said at least one of content and characteristics of said at least one message is determined to be stress-inducing if at least one of said content and said characteristics of said at least one message indicate that said at least one message is urgent and should not be delayed.

12. The recipient-aware message delivery system of claim 1, wherein said stress-aware message delivery engine is configured to process at least one message intended for delivery to the recipient user by analyzing content of said at least one message to identify at least one of a word, a phrasing, a sender, a subject, a capitalization, a punctuation, an included photographic image, an included video file, an included audio file, a length, and a timestamp of the message that is deemed to be indicative of a stress-inducing message.

13. The recipient-aware message delivery system of claim 1, wherein said stress-aware message delivery engine is configured to process at least one message intended for delivery to the recipient user by analyzing characteristics of said at least one message to identify at least one of a priority level and a priority flag associated with the message.

14. The recipient-aware message delivery system of claim 1, wherein said stress-aware message delivery engine is configured to delay if the recipient user is determined to be in the stressed state by waiting for a prescribed period of time before delivering said at least one message.

15. The recipient-aware message delivery system of claim 1, wherein said stress-aware message delivery engine is configured to delay if the recipient user is determined to be in the stressed state by waiting until the recipient user is determined not to be in the stressed state.

16. A computer-implemented method for recipient-aware message delivery, the computerized device comprising a memory operatively comprising a non-transitory data processor-readable medium, a data processor operative connected to the memory, sensor hardware operatively connected to the data processor and operable to capture recipient user activity data relating to a stress level of a message recipient, and user interface management instructions embodied in data processor-executable code stored in the memory, said user interface management instructions being executable by the data processor to provide a stress-aware message delivery engine, the method comprising:
observing recipient user activity via the sensor hardware;
capturing recipient user activity data via the sensor hardware;
analyzing the recipient user activity data to determine whether the recipient user is in a stressed state;
processing at least one message intended for delivery to the recipient user; and
selectively delaying delivery of said at least one message to the recipient as a function of the recipient’s stress level by:
delivering said at least one message without delay if the recipient user is determined not to be in the stressed state; and
delaying and delivering said at least one message after delay if the recipient user is determined to be in the stressed state.

17. The method of claim 16, wherein said capturing recipient user activity data via sensor hardware comprises capturing recipient user activity via at least one of a camera device, a microphone device, a biometric device, and an environmental sensor device.

18. The method of claim 16, wherein said analyzing recipient user activity data to determine whether the recipient user is in a stressed state comprises use of at least one of machine learning, facial recognition, voice data, biometric data, environmental data analysis.

19. The method of claim 16, wherein said processing at least one message intended for delivery to the recipient user comprises analyzing at least one of content and characteristics of said at least one message to determine whether said at least one message is stress-inducing.

20. The method of claim 19, wherein said delaying is performed only if said analyzing of said at least one of content and characteristics of said at least one message is determined to be stress-inducing.

21. The method of claim 19, wherein said delivering said at least one message without delay is performed even if said analyzing of said at least one of content and characteristics of said at least one message is determined to be stress-inducing if at least one of said content and said characteristics of said at least one message indicate that said at least one message is urgent and should not be delayed.

22. The method of claim 19, wherein said processing at least one message intended for delivery to the recipient user comprises analyzing content of said at least one message to identify at least one of a word, a phrasing, a sender, a subject, a capitalization, a punctuation, an included photographic image, an included video file, an included audio file, a length, and a timestamp of the message that is deemed to be indicative of a stress-inducing message.

23. The method of claim 19, wherein said processing at least one message intended for delivery to the recipient user comprises analyzing characteristics of said at least one message to identify at least one of a priority level and a priority flag associated with the message.

24. The method of claim 16, wherein said delaying is performed if the recipient user is determined to be in the stressed state by waiting for a prescribed period of time before delivering said at least one message.

25. The method of claim 16, wherein said delaying is performed if the recipient user is determined to be in the stressed state by waiting until the recipient user is determined not to be in the stressed state.

26. A computer program product for implementing a method for recipient-aware message delivery, the computer program product comprising a non-transitory computer-readable medium storing executable instructions that, when executed by a processor, cause a recipient-aware message delivery system to perform a method comprising:
observing recipient user activity via the sensor hardware;
capturing recipient user activity data via the sensor hardware;
analyzing the recipient user activity data to determine whether the recipient user is in a stressed state;
processing at least one message intended for delivery to the recipient user; and
selectively delaying delivery of said at least one message to the recipient as a function of the recipient’s stress level by:
delivering said at least one message without delay if the recipient user is determined not to be in the stressed state; and
delaying and delivering said at least one message after delay if the recipient user is determined to be in the stressed state.
